(19) United States
(12) Patent Application Publication
(10) Pub. No.: US 2021/0369580 A1
(43) Pub. Date: Dec. 2, 2021

(54) ANTIAGING MICROMOLECULAR METHOD

(71) Applicants: Luis M Fandos, Bay Shore, NY (US); Luis S Fandos, Bay Shore, NY (US)

(72) Inventors: Luis M Fandos, Bay Shore, NY (US); Luis S Fandos, Bay Shore, NY (US)

(21) Appl. No.: 16/883,488

(22) Filed: May 26, 2020

(51) Int. Cl. A61K 8/44; A61K 8/49; A61K 8/67; A61K 8/365; A61K 8/34; A61K 8/25; A61K 8/891; A61K 8/36; A61Q 19/08; A61Q 19/02; A61Q 19/00; A61Q 17/04

(52) U.S. Cl. CPC A61K 8/44; A61K 2800/884; A61K 8/671; A61K 8/365; A61K 8/347; A61K 8/25; A61K 8/891; A61K 8/361; A61Q 19/08; A61Q 19/02; A61Q 19/007; A61Q 17/04; A61K 2800/10; A61K 2800/92; A61K 8/4913

(57) ABSTRACT

A micromolecular organic method based on the mixed application of different active ingredients to provide an excellent corrective effect of the aesthetic and cosmetic alterations typical of aging. The active ingredients have synergistic effects, which potentiates or increases the effect on each other due the method of their combination. In addition to maintaining the good results achieved for a long time, the formulations, quantities, concentrations, sequencing, synergy, potentiation, amplification of effects, spatial structure effects relationship, form of administration, etc. present a wide margin of tolerance to the skin. The method including applying a BioNutrient MBM, a BioGel MBM, a BioExfoliant No. 1, a BioExfoliant No. 2, a cleaning mask, a face cleaner, a deep cleaner, a moisturizing lotion No. 1, a SPF lotion, a moisturizing lotion No. 2 with soothing effect and AntiSpot lotion to skin.

### BACKGROUND

The present invention generally relates to an antiaging method for skin. More specifically, the present invention relates to an antiaging method for skin using cosmetics.

The beginning of life evolution started on the Earth with the development of single-celled organisms. To form multicellular organisms, these primitive cells began to develop collagen fibers to promote strength and to form complex and specialized organisms. In all multicellular organisms, the collagen fibers are arranged in parallel bundles longitudinally aligned. This direction matches with the direction of the main effort of them. Therefore, the evolution of the species is not conceived without the fundamental contribution of the collagen fibers.

Nowadays, there are concerns of people regarding a healthy and personal appearance as it relates to the generalized aging of facial and body skin. The progressive increase in population and longevity is accompanied by skin aging in an increased number of people due to natural aging and a major exposure to air pollution. Thus, there is a desire of society is to improve their personal appearance, and to maintain it over time. This demand will tend to increase in the coming years. If we continue these trends, we can assume that aging and its consequences will soon became one of the main concerns of society. Society wishes have a means to correct skin deterioration and cosmetic and aesthetic alterations of natural skin aging. They no longer want to observe and demonstrate the cruel process of their own aging.

It is an object of the present invention to provide a method is to improve the quality of life of the elderly and the process of aging.

### SUMMARY

A micromolecular organic method based on the mixed application of different active ingredients to provide an excellent corrective effect of the aesthetic and cosmetic alterations typical of aging. The active ingredients have synergistic effects, which potentiates or increases the effect on each other due the method of their combination. In addition to maintaining the good results achieved for a long time, the formulations, quantities, concentrations, sequencing, synergy, potentiation, amplification of effects, spatial structure effects relationship, form of administration, etc. present a wide margin of tolerance to the skin. The method including applying a BioNutrient MBM, a BioGel MBM, a BioExfoliant No. 1, a BioExfoliant No. 2, a cleaning mask, a face cleaner, a deep cleaner, a moisturizing lotion No. 1, a SPF lotion, a moisturizing lotion No. 2 with soothing effect and AntiSpot lotion to skin.

### DETAILED DESCRIPTION

The present invention is antiaging macromolecular method affect the aging effects of skin. The antiaging macromolecular method provides for a stable and controlled method for facial and body skin rejuvenation. The antiaging macromolecular method provides for achieved improvements using the method can be maintained over the time. The antiaging macromolecular method is free of punctures, surgeries, and radiofrequency, among others invasive method. The antiaging macromolecular method provides for a method without toxic or foreign substances applied to the skin or the body. The antiaging macromolecular method provides for application at home.

The improvements achieved using this method due to the synergistic actions produced by the combination of the cosmetic peelings and liftings effects. The method provides long-term results. The cosmetic peelings of the method provide an innovative synergistic application form and active formulation of chemical peels with surface skin effects that are free of surgeries. From first applications, the cosmetic peelings of the method provide active ingredients to achieve excellent improvements in the visual and tactile aspect of the skin. The active ingredients of cosmetic peelings prepare the dermis or deep part of the skin by stimulating the cellular system of the dermis and fibroblasts for the action of a micromolecular component that will be applied on scheduled days. That is, the cosmetic peelings modify the surface of the skin with an excellence cosmetic peeling effect and high biological power.

The cosmetic lifting includes a micromolecular system where lifting effects are based on the application of Micromolecular Biological Material, (MBM). The MBM is composed by three amino acids, which include Glycine, Lysine and Proline. The cosmetic actives of the cosmetic lifting stimulate the increase of the population of dermis cells, which in turn generate fibroblasts. Fibroblasts synthesize all skin components such as type 1 collagen, amorphous matrix, and the others components of the dermis. These skin components, especially type 1 collagen, promote a lifting effect and the increase in tissue hydration in a stable and persistent way.

During experimentation of the method, it was investigated what the actions or influence of three specific biological materials on the stimulation of collagen biosynthesis, and the effects on the non-collagenous fibrous system of the skin, in a given range of concentrations. It was compared with others related biological elements which they interact at that level biological complexity in the body. It was determined that the effect of the specific biological material, also analyzing the appearance of new positive effects. The specific biological material is compounded by the three amino acids Lysine, Proline and Glycine. A new in Vitro line of research was created. A fully equipped laboratory of animal cell culture was installed. In addition, all the culture techniques, growth and collagen synthesis controls were developed. These investigations were conducted using a strict control of environmental conditions and using specific and non-specific related biological materials homologated in humans. The chemical indicator used was Syrian red to quantify the biosynthesis of type 1 collagen. The conclusion was that the cells of the tissues grown in Vitro reproduce the same functions as In Vivo tissues. It was determined that when specific biological material is added to the medium in culture cells, the effect on the activity the cells is the same as it could occur if MBM is ingested. It was determined that there can be long-term recover from damage of aging of all types of facial and body skin in all its evolutionary degrees. It was determined that there are dramatic results in a short period of time, with a long-term of the positive results. The method proposes a strategy consisting of the association of two practices of a lifting and peeling effect by using cosmetic actives. These practices are different, but complementary forms of applications. The functional biological assembly of the method is produced by a superficial chemical peeling effect and lifting effect. The lifting effect of the method uses an intense and prolonged stimulation of all parts of the dermis, especially fibroblasts, with the MBM amino acids of lysine, proline and glycine. This effect promotes an increase in fibroblast population or hyperplasia of them, and an increase of the activity and the quality of the synthesized molecules.

The method is a micromolecular organic method. The method is based on the mixed application of different active ingredients which provides an excellent corrective effect of the aesthetic and cosmetic alterations typical of aging. The active ingredients have synergistic effects, which potentiates or increases the effect on each other due the method of their combination. In addition to maintaining the good results achieved for a long time, the formulations, quantities, concentrations, sequencing, synergy, potentiation, amplification of effects, spatial structure effects relationship, form of administration, etc. present a wide margin of tolerance to the skin. The division into cycles also serves as a practical element for a better application and monitoring of the operation of the method. The most important fact is the achievement of an amplified sequential biological stimulation that can be maintained for years. The method is a simple, comfortable, predictable, gentle, and easy to execute and to control at home by the user. The method lacks undesirable side effects and on the contrary, it has additional beneficial effects to the body skin. The method use has shown the correction of facial and body skin aging without gender or age limits. The method can achieve great improvements in all types of natural skin and skin conditions, produced by natural deterioration of aging. It is believe the method is a revolutionary contribution to the present and future of cosmetics that can be applied as a cosmetic treatment at home as well as professional skin treatment facilities.

The method includes the use of an organic micromolecular kit of treatment materials. All the cosmetic actives, nutrients and elements required to apply the method are included in the kit. The kit was designed to satisfy all the needs that arose during its application. The treatment materials can also be purchase separately. To verify the legitimacy of the treatment materials, an authenticity stamp is used so that the user can check to be assured the treatment material contains the proper ingredients to be used with the method. The kit includes a BioNutrient MBM, a BioGel MBM, a BioExfoliant No. 1, a BioExfoliant No. 2, a cleaning mask, a face cleaner, a deep cleaner, a moisturizing lotion No. 1, a SPF (Sun Protection Factor) lotion, a moisturizing lotion No. 2 with soothing effect and AntiSpot lotion. Accessories to be used with the method are cosmetic brushes No. 1, 2, 3 and 4, sponges, liners and a pen.

The MBM BioNutrient composition includes Glycine, Lysine and Proline. Glycine has a Systematic IUPAC name of 2-Aminoethanoic acid and the Chemical formula of C2H5NO2. Lysine has a Systematic IUPAC name of 2,6-diaminohexanoic acid and Chemical formula of C6H14N2O2. Proline has a Systematic IUPAC name of Pyrrolidine-2-carboxylic acid and a Chemical formula of C5H9NO2. The MBM BioNutrient composition is mixed in a ratio of 2 of 4 parts Glycine, 1 of 4 parts Lysine and 1 of 4 parts Proline. The BioGel MBM contains the same amino acids as the BioNutrient MBM of Glycine, Lysine and Proline. The BioGel MBM further includes a suspension in a liposome water-soluble cosmetic gel for facial application. The BioExfoliant No. 1 is an active ingredient diluted with inert component(s) to make up the desired concentration. The active ingredient is 1% Retinoic acid having the chemical formula of C20H28O2 and IUPAC name of (2E,4E,6E,8E)-3,7-dimethyl-9-(2,6,6-trimethylcyclohexen-1-yl)nona-2,4,6,8-tetraenoic acid. The retinoic acid at 1% is diluted with inert component(s) to make up the desired concentration (1%). This is so bioexfoliant has only retinoic acid with no other active ingredients. One example is the use of Bentonite as the inert component. BioExfoliant No. 2 is an active ingredient diluted with inert component(s) to make up the desired concentration. The active ingredient is 25% glycolic acid having the chemical formula: C2H4O3 and IUPAC name of 2-Hydroxyethanoic acid. The glycolic acid at 25% is diluted with inert component(s) to make up the desired concentration (25%). This is so bioexfoliant has only glycolic acid with no other active ingredients. One example is the use of Bentonite as the inert component. The cleaning mask is a lotion with an active ingredient diluted with inert component(s) to make up the desired concentration on the mask. The active ingredient is 0.05% isotretinoin having the chemical formula: C20H28O2 and IUPAC name of (2Z,4E,6E,8E)-3,7-Dimethyl-9-(2,6,6-trimethylcyclohex-1-en-1-yl)nona-2,4,6,8-tetraenoic acid. The isotretinoin at 0.05% is diluted with inert component(s) to make up the desired concentration (0.05%). This is so the cleaning mask has only isotretinoin with no other active ingredients. One example is the use of Caprylic as the inert component. The face cleaner includes royal jelly and a betainic derivative in a 50/50 ratio. The deep cleaner includes 3 parts of 6 parts Kaolin, 2.4 parts of 6 parts Bentonite and 0.6 parts of 6 parts Zinc. Moisturizing lotion No. 1 includes 5 parts to 10 parts Sweet Almond Oil, 0.5 parts to 10 parts Vitamin E, 3 parts to 10 parts Honey and 1.5 parts to 10 parts Royal Jelly. The SPF (Sun Protection Factor) lotion includes Cetyl dimethicone copolyol, cyclomethicone, octyl ethoxy cinnamate, benzophenone titanium oxide, tocopheryl acetate, ascorbyl palmitate, bht, sodium citrate mnf, silk protein, dimethicone, 2-bromo-2-nitropropane-1,3 diol-ethyl paraben, marigold officinalis extract, rosehip extract. For example SPF 110 lotion has Avobenzone 3%, homosalate 15%, octisalate 5%, octocrylene 10%, oxybenzone 6% there all distributed in equal parts. Moisturizing lotion No. 2 includes ⅓ Chamomile extract, ⅓ marigold extract, and ⅓ mallow extract. Antispot lotion includes an active ingredient diluted with inert component(s) to make up the desired concentration. The active ingredient is 2% hydroquinone. The hydroquinone at 2% is diluted with inert component(s) to make up the desired concentration (2%). This is so the antispot lotion has only hydroquinone with no other active ingredients. One example is the use of Dimethicone as the inert component.

The method employs the treatment materials of the kit and guide to the steps of the method. The user should write down in a daily calendar notebook the day by day practices and applications of the method. It is also advisable to log any discomforts and other sensations the user encounters and all the changes observed in the users skin. The user should also take and save photos taken of the user. This provides monitoring of the results achieved. The guide and the daily calendar can be used for any questions the user may have during the use of the method. The method is compounded by 2 cycles of application of 90 days each. It is still possible to continue its application for a longer time.

Day 1 of the method includes the following. The user washes their face with clean water using small wet sponge with eyes closed. All washing of and applications to the face includes the areas of face, neck and neckline. Use the sponge in circular motion on the face. Dry skin using a dry towel. Take pictures of entire face full front and then degree angle from each side of the face. Clean face using face cleaner using circular motion with eyes closed. Apply deep cleaner using brush #4 using a circular motion starting from the center of the face and work your way outwards. The brush can be a small brush or any cosmetic brush. Leave deep cleaner on face for 20 min. Dry face using a fan or blow dryer. Wash your face with wet sponge of water using circular motion from center to side of the face, while keeping eyes closed. Apply a thin layer of bioexfoliant No. 1 using brush No. 1. Leave bioexfoliant No. 1 on face for 20 min. Use a fan or blow dryer with cool air on face while keeping eyes closed. Wash face using a wet sponge of water in a circular motion from inside out, while keeping eyes closed. Dry face using a towel. Apply with a circular motion moisturizing lotion No. 1 and then SPF LOTION with rating of 80-110. Repeat the set of applying moisturizing lotion No. 1 and then SPF LOTION with rating of 80-110 in a double layer 3 times a day for 4 days. Then, a single layer 3 times a day. During the use of the method it is advised to avoid immediate and direct exposure to the sun. Make an observations in a diary calendar.

DAY 2, before going to bed perform the following. Clean face with face cleaner. Apply the cleaning mask every night with Brush No. 3 in a circular motion. Leave the mask on overnight and remove it in the morning with a wet sponge of water. Wash your face with clean water until all traces of the cleaning mask are removed. Repeat the application of the cleaning mask for 6 consecutive nights, that is, from the night of day 2 to the night of day 7. On day 6, repeat the process of day 1.

DAY 8 perform the following. In the morning clean the face with face cleaner. Apply BioGel MBM in a circular motion. Apply daily on face and body for 21 days, that is, for 3 consecutive weeks from day 8 to day 28 of the treatment, inclusive, between 6 to 8 pm. Apply Sun Protection Factor (SPF LOTION). Ingest MBM BioNutrient capsule with a little water, juice, yogurt or milk. Ingest the MBM BioNutrient from day 8 to day 28 inclusive, that is, for 3 consecutive weeks. In the afternoon when at least 7 hours has pasted, the user cleans face with face cleaner. Cleaning face and application of BioGel MBM should be for 3 consecutive weeks. Ingest the MBM BioNutrient for 3 consecutive weeks. DAY 12 perform the following. Repeat steps of Day 1. DAY 15 apply the Moisturizing lotion No. 1 from day 15 to day 77 before going to bed.

DAY 17 perform the following. Clean face with face cleaner. Mask face with BioExfoliant No. 2 with Brush No. 2. Allow BioExfoliant No. 2 on face for 15 minutes while fanning or drying the face with fresh and dry air while the mask is on face. Remove BioExfoliant No. 2 with a wet sponge of water. Wash face with clean water until all traces of BioExfoliant No. 2 are removed, while keeping eyes closed. Dry the skin with a clean dry towel starting with the eyes, then move outward to rest of face, neck and neckline, keeping eyes closed. Apply Moisturizing lotion No. 1 and SPF LOTION with at least 80 rating to skin. Remember to apply a double layer 3 times a day for the next consecutive 10 days. Then, continue the application with a single layer 3 times per day. In case of skin irritation, intense itching or heat sensation after application of Bioexfoliant No. 2 and/or by prolonged exposure to the sun, shield or apply fresh air for a few minutes until the discomfort disappears. If discomfort continues, apply a thin layer of Moisturizing lotion No. 2 and Antispot lotion to the affected areas.

DAY 30 perform the following. Clean face with face cleaner. Clean face with deep cleaner using Brush No. 4. Allow deep cleaner on face for 20 minutes, fanning or drying face with fresh dry air. Remove deep cleaner with a wet sponge of water. Wash face with clean water until all traces of deep cleaner are removed, keeping eyes closed. This mask produces a pleasant sensation of freshness and tension of the skin. Dry skin with a clean and dry towel starting with the eyes and then, the rest of your face, neck and neckline with eyes closed. Apply Moisturizing lotion No. 1 and SPF LOTION with at least 80 rating.

DAY 36 perform the following. In the morning and afternoon, Repeat the steps of DAY 8 from day 36 to day 56 inclusive, that is, for 3 consecutive weeks. Allow at least 7 hours between morning and afternoon. DAY 37, in the afternoon, repeat the afternoon steps of DAY 17. DAY 60, in the afternoon, repeat the afternoon steps of DAY 30.

DAY 64 perform the following. Clean face with face cleaner and take PARAMETRIC SELFIES. Record observations in Diary Calendar, prior to morning steps. In the morning and afternoon repeat steps for DAY 8, from day 64 to day 84 inclusive, that is, for 3 consecutive weeks. DAY 85 at night before going to the bed, repeat steps for DAY 2. Apply cleaning mask with brush No. 3. Repeat this application for 6 consecutive nights, that is, from the night of day 85 to the night of day 90. After 90 days the 1st Application Cycle is done and it is recommended to rest 10 days before starting the 2^{nd} application Cycle of the same method. To avoid mistakes on what day it is, it is recommended to indicate your routine in the daily calendar.

In summary of the application of the method, the Moisturizing lotion No. 1 and FPS are apply a single layer with your fingers by circular movements 3 times per day at 7 am, 11 am and 3 pm every day of both cycles. Apply a double layer of the Moisturizing lotion No. 1 and FPS 3 times a day at 7 am, 11 am and when you have to apply the BioExfoliant No. 1 (with its 4 subsequent days), and BioExfoliant No. 2 (with its 10 subsequent days). In case of direct exposure to the sun, protect the skin using a hat. Moisturizing lotion No. 1 should also be applied at night before going to bed from day 15, until day 77. In case of dehydrated skin, more applications can be made. The daily application of Moisturizing lotion No. 1 can be applied as necessary (e.g. dry or dehydrated skin). The deep cleaner BioExfoliant No. 1, BioExfoliant No. 2 and cleaning mask are applied using a specific brush. Apply a double layer, leaving to dry 3 minutes between applications, while fanning or drying your face with fresh and dry air. Remove them with a wet sponge with water. Wash your face with clean water until all traces of them are removed. Your eyes must always be closed. Cleaning mask requires to leave it acting during the night and to be removed in the morning. The BioNutrient MBM and BioGel MBM will be administered together in sequences of 3-week, followed by a rest period. The BioGel MBM will be applied on your face and body skin surface, and the BioNutrient will be administered orally. By doing so, their benefits are potentiated. It is advisable to administer them for years. PARAMETRIC SELFIES are as follows. Photographic shots will always be taken with the same background, light and distance, to compare them and to see how your face evolves. You should record the date of the photo in the Diary Calendar notebook. Observe scheme in the Diary Calendar. It is advisable to take a photo every 30 (thirty) days. Take them previous cleaning your face with face cleaner and before starting the rest of the applications of that day. It is important to take the first shot before the start of the cycle (Day 1), and at the end of each cycle (Day 90 and 180).

Tips on using the method. Before making any application, you should clean your hands with gel alcohol. After using the brushes, wash them with plenty of clean water until removing all traces of the product. Your eyes must always be closed when removing the products with sponges. Wash your face with plenty of water without strongly rubbing your skin. Dry with a dry, clean and soft towel without scrubbing. To avoid irritation, rest a towel on your face and let it absorb all the water. Some traces of cosmetics, and especially, SPF LOTION are very irritating to the eyes, but they never cause any kind of injury. They can give an intense burning sensation, which quickly disappear by washing with plenty of cold water. In cases of skin irritation, apply Moisturizing lotion No. 1 to soothe burning, itching, or redness. Moisturizing lotion No. 1 can be applied every 8 hours. In cases your skin is prone to produce skin spots, and when the UVB is high or hot weather, apply a layer of Anti spot lotion and repeat every 12 hours for 10 to 15 days. Apply a double layer of high-grade photoprotection (SPF LOTION) when applying the BioExfoliant No. 1 and its 4 subsequent days, the BioExfoliant No. 2 (and its 10 subsequent days), 3 times per day (at 7, 11 and 15 hours). Then, you can apply a single layer of a moderate photoprotection three times a day. AVOID sun exposure, heat sources, irritants, waxing and shaving, application of scrubs or other cabinet practices, and the use of appliances for aesthetic purposes. USE A HAT if you are exposed to direct sun.

While different embodiments of the invention have been described in detail herein, it will be appreciated by those skilled in the art that various modifications and alternatives to the embodiments could be developed in light of the overall teachings of the disclosure. Accordingly, the particular arrangements are illustrative only and are not limiting as to the scope of the invention that is to be given the full breadth of any and all equivalents thereof

I claim:

1. A antiaging macromolecular method, said method comprising applying a BioNutrient MBM, a BioGel MBM, a BioExfoliant No. 1, a BioExfoliant No. 2, a cleaning mask, a face cleaner, a deep cleaner, a moisturizing lotion No. 1, a SPF lotion, a moisturizing lotion No. 2 with soothing effect and AntiSpot lotion to skin.

2. The method of claim 1, wherein the MBM BioNutrient composition includes Glycine, Lysine and Proline, the BioGel MBM includes Glycine, Lysine and Proline in a suspension of a liposome water-soluble cosmetic gel, the BioExfoliant No. 1 includes 1% Retinoic acid as an active ingredient diluted with at least one inert component, the BioExfoliant No. 2 includes 25% glycolic acid as an active ingredient diluted with at least one inert component, the cleaning mask includes 0.05% isotretinoin as an active ingredient diluted with at least one inert component, the deep cleaner includes Kaolin, Bentonite and Zinc, the Moisturizing lotion No. 1 includes Sweet Almond Oil, Vitamin E, Honey and Royal Jelly, the Moisturizing lotion No. 2 includes Chamomile extract, marigold extract, and mallow extract, and the Antispot lotion includes 2% hydroquinone as an active ingredient diluted with at least one inert component.

3. The method of claim 2, wherein the MBM BioNutrient composition is mixed in a ratio of 2 of 4 parts Glycine, 1 of 4 parts Lysine and 1 of 4 parts Proline; where in the BioGel MBM contains the same amino acids rations as the BioNutrient MBM of Glycine, Lysine and Proline suspended in a liposome water-soluble cosmetic gel; wherein the face cleaner includes royal jelly and a betainic derivative in a 50/50 ratio; wherein the deep cleaner includes 3 parts of 6 parts Kaolin, 2.4 parts of 6 parts Bentonite and 0.6 parts of 6 parts Zinc; wherein the Moisturizing lotion No. 1 includes 5 parts to 10 parts Sweet Almond Oil, 0.5 parts to 10 parts Vitamin E, 3 parts to 10 parts Honey and 1.5 parts to 10 parts Royal Jelly, and wherein the Moisturizing lotion No. 2 includes ⅓ Chamomile extract, ⅓ marigold extract, and ⅓ mallow extract.

4. The method of claim 3, where the BioExfoliant No. 1 includes Bentonite; wherein the BioExfoliant No. 2 includes Bentonite; wherein the cleaning mask includes Caprylic, and wherein the Antispot lotion includes Dimethicone.

5. The method of claim 1, wherein the method is performed with the following steps comprising of:
Starting Day 1, washing skin with clean water using small wet sponge, dry skin using a dry towel, applying face cleaner to the skin, applying deep cleaner to the skin using a brush, leaving deep cleaner on skin for at least 20 min and allowing skin to dry, washing skin with wet sponge of water, applying a layer of bioexfoliant No. 1 using a brush, leaving bioexfoliant No. 1 on skin for at least for 20 min and allowing skin to dry, washing skin using a wet sponge of water, dry skin using a towel, applying moisturizing lotion No. 1 to skin, applying SPF lotion to the skin with at least a rating of 80-110, repeat the applying of moisturizing lotion No. 1 and SPF lotion in a double layer 3 times a day for 4 days and after 4 days in a single layer 3 times a day;
starting DAY 2 before going to bed perform the following, cleaning skin with face cleaner, applying cleaning mask to skin every night with Brush, leaving the cleaning mask on overnight and removing the cleaning mask the next morning with a wet sponge of clean water until the cleaning mask is removed, repeating the application of the cleaning mask for 6 consecutive nights which is from the night of day 2 to the night of day 7, on day 6, repeat the steps of day 1;
starting DAY 8 in the morning cleaning the skin with face cleaner and applying BioGel MBM to skin for 21 days which is from day 8 to day 28 of the method, applying SPF lotion after the BioGel MBM, then ingesting the MBM BioNutrient, in the afternoon when at least 7 hours has passed from morning cleaning skin with face cleaner, on DAY 12 repeating steps of Day 1, on DAY 15 applying the Moisturizing lotion No. 1 from day 15 to day 77 before going to bed;
starting DAY 17 cleaning skin with face cleaner, masking skin with BioExfoliant No. 2 using a Brush, allowing the BioExfoliant No. 2 on skin for at least 15 minutes while allowing skin to dry, after the 15 minutes removing the BioExfoliant No. 2 with a wet sponge of water by washing the skin with clean water until the BioExfoliant No. 2 is removed, drying the skin after washing, applying the Moisturizing lotion No. 1 and SPF lotion, whereby a double layer 3 times a day for the next consecutive 10 days is applied, then continue the application with a single layer 3 times per day, In case of skin irritation, apply a thin layer of Moisturizing lotion No. 2 and Antispot lotion to the affected areas;
Starting DAY 30 clean skin with face cleaner, clean skin with deep cleaner using a Brush, allow deep cleaner on skin for at least 20 minutes while allowing skin to dry, remove deep cleaner with a wet sponge of water by washing the skin with clean water until the deep cleaner is removed, drying skin, Moisturizing lotion No. 1 and SPF lotion;
starting DAY 36 in the morning and afternoon repeat the steps of DAY 8 from day 36 to day 56 while allowing at least 7 hours between morning and afternoon, on DAY 37 in the afternoon repeat the afternoon steps of DAY 17 and on DAY 60 in the afternoon repeat the afternoon steps of DAY 30; and
starting DAY 64 clean the skin with face cleaner, in the morning and afternoon repeat steps for DAY 8, from day 64 to day 84, on DAY 85 at night before going to the bed, repeat steps for DAY 2, apply cleaning mask with brush and repeat this application for 6 consecutive nights to Day 90.

6. The method of claim 2, wherein the method is performed with the following steps comprising of:
Starting Day 1, washing skin with clean water using small wet sponge, dry skin using a dry towel, applying face cleaner to the skin, applying deep cleaner to the skin using a brush, leaving deep cleaner on skin for at least 20 min and allowing skin to dry, washing skin with wet sponge of water, applying a layer of bioexfoliant No. 1 using a brush, leaving bioexfoliant No. 1 on skin for at least for 20 min and allowing skin to dry, washing skin using a wet sponge of water, dry skin using a towel, applying moisturizing lotion No. 1 to skin, applying SPF lotion to the skin with at least a rating of 80-110, repeat the applying of moisturizing lotion No. 1 and SPF lotion in a double layer 3 times a day for 4 days and after 4 days in a single layer 3 times a day;
starting DAY 2 before going to bed perform the following, cleaning skin with face cleaner, applying cleaning mask to skin every night with Brush, leaving the cleaning mask on overnight and removing the cleaning mask the next morning with a wet sponge of clean water until the cleaning mask is removed, repeating the application of the cleaning mask for 6 consecutive nights which is from the night of day 2 to the night of day 7, on day 6, repeat the steps of day 1;
starting DAY 8 in the morning cleaning the skin with face cleaner and applying BioGel MBM to skin for 21 days which is from day 8 to day 28 of the method, applying SPF lotion after the BioGel MBM, then ingesting the MBM BioNutrient, in the afternoon when at least 7 hours has passed from morning cleaning skin with face cleaner, on DAY 12 repeating steps of Day 1, on DAY 15 applying the Moisturizing lotion No. 1 from day 15 to day 77 before going to bed;
starting DAY 17 cleaning skin with face cleaner, masking skin with BioExfoliant No. 2 using a Brush, allowing the BioExfoliant No. 2 on skin for at least 15 minutes while allowing skin to dry, after the 15 minutes removing the BioExfoliant No. 2 with a wet sponge of water by washing the skin with clean water until the BioExfoliant No. 2 is removed, drying the skin after washing, applying the Moisturizing lotion No. 1 and SPF lotion, whereby a double layer 3 times a day for the next consecutive 10 days is applied, then continue the application with a single layer 3 times per day, In case of skin irritation, apply a thin layer of Moisturizing lotion No. 2 and Antispot lotion to the affected areas;
Starting DAY 30 clean skin with face cleaner, clean skin with deep cleaner using a Brush, allow deep cleaner on skin for at least 20 minutes while allowing skin to dry, remove deep cleaner with a wet sponge of water by washing the skin with clean water until the deep cleaner is removed, drying skin, Moisturizing lotion No. 1 and SPF lotion;
starting DAY 36 in the morning and afternoon repeat the steps of DAY 8 from day 36 to day 56 while allowing at least 7 hours between morning and afternoon, on DAY 37 in the afternoon repeat the afternoon steps of DAY 17 and on DAY 60 in the afternoon repeat the afternoon steps of DAY 30; and
starting DAY 64 clean the skin with face cleaner, in the morning and afternoon repeat steps for DAY 8, from day 64 to day 84, on DAY 85 at night before going to the bed, repeat steps for DAY 2, apply cleaning mask with brush and repeat this application for 6 consecutive nights to Day 90.

7. The method of claim 6, wherein the MBM BioNutrient composition is mixed in a ratio of 2 of 4 parts Glycine, 1 of 4 parts Lysine and 1 of 4 parts Proline; where in the BioGel MBM contains the same amino acids rations as the BioNutrient MBM of Glycine, Lysine and Proline suspended in a liposome water-soluble cosmetic gel; wherein the face cleaner includes royal jelly and a betainic derivative in a 50/50 ratio; wherein the deep cleaner includes 3 parts of 6 parts Kaolin, 2.4 parts of 6 parts Bentonite and 0.6 parts of 6 parts Zinc; wherein the Moisturizing lotion No. 1 includes 5 parts to 10 parts Sweet Almond Oil, 0.5 parts to 10 parts Vitamin E, 3 parts to 10 parts Honey and 1.5 parts to 10 parts Royal Jelly, and wherein the Moisturizing lotion No. 2 includes ⅓ Chamomile extract, ⅓ marigold extract, and ⅓ mallow extract.

8. The method of claim 7, where the BioExfoliant No. 1 includes Bentonite; wherein the BioExfoliant No. 2 includes Bentonite; wherein the cleaning mask includes Caprylic, and wherein the Antispot lotion includes Dimethicone.
